(19) United States
(12) Patent Application Publication
(10) Pub. No.: US 2026/0226531 A1
(43) Pub. Date: Aug. 6, 2026

(54) 50K LIQUID-PHASE CHIP FOR PIGS BASED ON MULTIPLE SINGLE NUCLEOTIDE POLYMORPHISMS

(71) Applicant: CHINA AGRICULTURAL UNIVERSITY, Beijing (CN)

(72) Inventors: Xiangdong DING, Beijing (CN); Huatao LIU, Beijing (CN); Hehe DU, Beijing (CN); Zipeng ZHANG, Beijing (CN); Qin ZHANG, Beijing (CN); Chuduan WANG, Beijing (CN)

(73) Assignee: CHINA AGRICULTURAL UNIVERSITY, Beijing (CN)

(21) Appl. No.: 19/394,946

(22) Filed: Nov. 20, 2025

Related U.S. Application Data
(63) Continuation-in-part of application No. 18/935,640, filed on Nov. 3, 2024, now abandoned.
Application No. 18/935,640 is a continuation of application No. PCT/CN2023/127964, filed on Oct. 30, 2023.

(30) Foreign Application Priority Data
May 17, 2023 (CN) 202310552851.6

(51) Int. Cl. C12Q 1/6837; C12Q 1/6869; C12Q 1/6888; C40B 40/06; C40B 50/00

(52) U.S. Cl. CPC C12Q 1/6837; C12Q 1/6869; C12Q 1/6888; C40B 40/06; C40B 50/00; C12Q 2600/124; C12Q 2600/156; C12Q 2600/16

(57) ABSTRACT

The present disclosure relates to the field of genetic molecular breeding, specifically to a 50K liquid-phase chip for pigs based on multiple single nucleotide-polymorphism (mSNP) and its application. The DNA probe design of the chip in the present disclosure takes into account the distribution of captured SNP loci across the genome, the polymorphism of the loci, the quality of mSNP markers, and other issues, effectively avoiding problems such as uneven marker density and poor polymorphism. It also considers the quality of mSNP markers and the issue of linkage disequilibrium among mSNP markers. While adhering to the basic principles of liquid-phase chip design, genomic regions with moderate linkage disequilibrium between markers were selected, generating more SNP markers with high genotyping quality and moderate linkage disequilibrium within the probe region with the target loci. The mSNP liquid-phase chip of the present disclosure expands the detectable number of SNPs to 1.5-2 times that of the target loci, addressing the issue of the relatively small number of high-quality mSNP markers in traditional liquid-phase chips without increasing costs.

### CROSS-REFERENCE TO RELATED APPLICATION

This application is a continuation-in-part application of U.S. patent application Ser. No. 18/935,640 filed on Nov. 3, 2024, which is a Continuation of International Application No. PCT/CN2023/127964, filed Oct. 30, 2023, which claims priority to Chinese Patent Application No. 202310552851.6, filed on May 17, 2023, the entire contents of each of which are hereby incorporated by reference.

### SEQUENCE LISTING

The instant application contains a Sequence Listing which has been submitted electronically in XML format and is hereby incorporated by reference in its entirety. The XML copy, created on Apr. 16, 2026, is named “2026-04-16-sequence listing-6A801-H001US01”, and is 78,791,674 bytes in size.

### TECHNICAL FIELD

The present disclosure relates to the field of genetic molecular breeding, specifically to a 50K liquid-phase chip for pigs based on multiple single nucleotide polymorphism (mSNP) technologies, more specifically a 50K mSNP liquid-phase chip for pigs.

### BACKGROUND

Single nucleotide polymorphism (SNP) is characterized by its large number, wide distribution across the genome, ease of large-scale rapid screening, and genotyping, making it the best molecular marker available today. The rapid development of molecular detection technology has given rise to SNP chips capable of high-throughput genotyping. At present, the mainstream SNP chips on the market are mainly developed based on solid-phase technology, with relatively high detection accuracy. As shown in FIG. 1, solid-phase chips primarily perform multiple detections on target loci to ensure the accuracy of genotyping. However, solid-phase chips have disadvantages such as poor flexibility, strict sample size requirements (must be a multiple of 12 or 24), and high customization costs, limiting their large-scale use in practical breeding.

Unlike solid-phase chips, liquid-phase chips based on genotyping by target sequencing (GBTS) technology have advantages such as easy addition or removal of markers and no sample size requirements. FIG. 1 shows that GBTS technology mainly performs multiple sequencing of the target loci and its upstream and downstream regions to ensure the quality of target loci genotyping. Unlike solid-phase chips, liquid-phase chips can also genotype polymorphic loci upstream and downstream of the target loci, known as multiple single nucleotide polymorphism clusters (mSNPs or multiple dispersed nucleotide polymorphisms, MNPs). The mSNPs centered on target loci are tightly linked and are in a state of high linkage disequilibrium. In many species, the genotypes of mSNPs upstream and downstream of the target loci are considered consistent with the target loci and do not provide additional information. As shown in FIG. 2, the linkage disequilibrium (r²) within a 200 bp fragment of multiple rice varieties is 1, indicating that the SNP genotypes within these fragments are linked and identical. Even if there are multiple mSNPs, the information provided is the same as that of a single target locus. Therefore, although liquid-phase chips can detect mSNPs exceeding the number of target loci, the mSNP information upstream and downstream of the target loci is rarely used, and genetic analysis and molecular breeding mainly focus on the target loci.

Although high linkage disequilibrium between closely linked markers exists in animal genomes, due to the high diversity of animal genomes and high average heterozygosity of markers, there are many genomic regions where the degree of linkage disequilibrium between markers is moderate. For liquid-phase chips, markers upstream and downstream of the target loci can provide additional information. Therefore, multiple single nucleotide polymorphism technology can clearly increase the number of effective mSNP markers without increasing the number of target loci markers, thereby enhancing the information content of liquid-phase chips and fully utilizing the characteristics of GBTS technology; this is not attainable with solid-phase microarray technologies.

Although multiple single nucleotide polymorphism technology increases mSNP markers, there are many challenges in utilizing this marker information. Directly using mSNPs as single markers often introduces noise due to high linkage disequilibrium between markers, reducing the effectiveness of genetic analysis. Haplotype analysis can simultaneously utilize information from multiple SNP markers, improving the effectiveness of genetic analysis. However, many studies have shown that if the marker spacing is too large, the low linkage disequilibrium between markers can result in numerous haplotypes, which not only fails to increase the power of genetic analysis but also adds complexity to the analysis and increases computation time. Currently, the mainstream 50K SNP chips for pigs, such as the SNP chip Porcine GGP 50K designed by Neogen Corporation (containing 50,697 SNP markers), have an average marker spacing of 40 kb and an average linkage disequilibrium of 0.2. Theoretical research and breeding practices have shown that haplotype analysis with a fixed segment length or a fixed number of SNPs cannot improve the accuracy of genomic selection. For liquid-phase chips, although the average spacing and linkage disequilibrium level of target loci are similar to those of the SNP chip Porcine GGP 50 K, mSNP markers upstream and downstream of the target loci have much smaller spacing, greatly enhancing the degree of linkage disequilibrium between markers. Treating them as a block for haplotype analysis can significantly improve the accuracy of genomic selection and the effectiveness of genetic analysis.

### SUMMARY

The present disclosure has developed a 50K liquid-phase chip for pigs and also provides methods for chip target loci development and analysis. Using the chip and analysis strategy designed by the present disclosure maximizes the use of mSNP marker information upstream and downstream of the target loci, improving the efficiency of genetic analysis and molecular breeding in pigs.

The present disclosure provides a probe hybridization solution for genotyping pigs, comprising a set of isolated, synthetic DNA molecules, wherein the set of DNA molecules is configured to target a plurality of target single nucleotide polymorphism (SNP) loci, which are related to pig breeds.

In some embodiments, the plurality of target SNP loci are identified by: aligning whole-genome sequencing data of Duroc, Large White, and Landrace pig breeds, selecting genomic regions with moderate linkage disequilibrium between markers, and screening and determining selected sites as the target SNP loci.

In some embodiments, the genotyping quality of the target SNP loci meets the following criteria: a missing rate of NA<0.1, a minimum allele frequency (MAF)≥0.05, and heterozygosity (Het)<0.5.

In some embodiments, the principles for selecting the target SNP loci are: (a) uniform distribution across chromosomes, with denser distribution at both ends of the chromosomes; (b) polymorphism considers MAF>0.35 in Duroc, Landrace, and Large White pigs; (c) average linkage disequilibrium (r²) with upstream and downstream SNP markers less than 0.85; (d) comparison with the QTLdb database, aiming for SNP markers to be located in QTL regions related to economic traits; (e) overlap with some loci of the known 50K chip for pigs.

In some embodiments, the known 50K chip for pigs is a 50K SNP liquid-phase chip, GGP50K from Neogen Corporation, or Zhongxin No. 1.

In some embodiments, the set of DNA molecules is determined with multiple single nucleotide polymorphism (mSNP) technologies, and each target SNP locus is targeted by 1-4 DNA molecules in the set of DNA molecules.

In some embodiments, a length of each of the DNA molecules is 110 base pairs.

In some embodiments, the set of DNA molecules comprises sequences set forth in SEQ ID NOs: 30,687, 30,697, 30,702, 30,711, 30,722, 30,725, 30,764, 30,773, 30,775, 30,782, 30,783, 30,799, 30,823, 30,855, 30,862, 30,887, 30,910, 30,911, 30,932, 30,933, 30,938, 30,959, and 30,960.

In some embodiments, the set of DNA molecules comprises sequences set forth in SEQ ID NOs: 1-80,631.

In some embodiments, a concentration of the DNA molecules in the probe hybridization solution is 1-5 pmol/ml.

In some embodiments, the probe hybridization solution comprises a buffer solution, which is a mixture of EDTA and Tris-HCl.

In some embodiments, for a total volume of 500 ml, the probe hybridization solution further includes:

| Component name | Quantity |
|---|---|
| Pooled, barcoded library | 0.6 μL |
| GenoBaits Block I | 5 μL |
| GenoBaits Block II | 2 μL |
| forILM/MGI | |
| The DNA molecules | 300 ng |

In some embodiments, the probe hybridization solution is concentrated to dryness using a vacuum concentrator at a temperature≤60° C.

The present disclosure provides a liquid-phase chip, comprising the probe hybridization solution.

The present disclosure provides a method for 50K mSNP marker selection and the probe hybridization solution preparation for a 50K liquid-phase chip used for multiple single nucleotide-polymorphism (mSNP), which utilizes whole-genome sequencing data of pig breeds to mine and screen target SNP loci, then designs and optimizes the set of DNA molecules (i.e., probes) for the target SNP loci, ultimately resulting in the determination of the set of DNA molecules. The method includes the following steps:
Step 1, determining target SNP loci: based on whole-genome sequencing data from Duroc, Large White, and Landrace, aligning to the pig reference genome, screening out genomic regions exhibiting moderate linkage disequilibrium between markers, screening the target SNP loci from the genomic regions.
Step 2, designing the DNA molecules (i.e., probes) based on the determined target SNP loci: a length of each of DNA molecules is 110 base pairs, for each of the target SNP loci, utilize multiple single nucleotide polymorphism (mSNP) technologies to design 1-4 DNA molecules which cover a 165 base pair region centered on the target SNP loci. The principles for DNA molecules design are: 1) select the DNA molecules with a content between 30% and 80%; 2) choose regions with a number of homologous areas≤5; 3) select the DNA molecules regions that do not contain SSR, N regions.
Step 3, selecting and optimizing DNA molecules containing high-quality mSNPs: DNA molecules are hybridized and sequenced, and the genotyping quality of mSNPs, including the target SNP loci, is detected. Set a missing rate of NA<0.1, a minimum allele frequency (MAF)≥0.05, and heterozygosity (Het)<0.5 as standards to screen mSNPs, removing those that do not meet the standards. If the DNA molecules does not meet the genotype quality control requirements of mSNPs, delete the DNA molecules and the corresponding target SNP loci, redesign new DNA molecules according to Steps 1 and 2, and continue to test and optimize the DNA molecules as per this step. The mSNPs that meet the quality inspection requirements are finally used as the target loci of the 50K mSNP liquid-phase chip.

In some embodiments, the pig reference genome is the pig reference genome Sscrofa11.1 (GenBank: GCA_000003025.6; RefSeq: GCF_000003025.6); the principles for screening target SNP loci are: (1) uniform distribution across chromosomes, with denser distribution at both ends of the chromosomes; (2) polymorphism considers MAF>0.35 in Duroc, Landrace, and Large White pigs; (3) average linkage disequilibrium (r²) with upstream and downstream SNP markers less than 0.85; (4) comparison with the QTLdb database, aiming for SNP markers to be located in QTL regions related to economic traits; (5) overlap with some loci on the known 50K chip for pigs.

In some embodiments, the known 50K chip for pigs is a 50K SNP liquid-phase microarray, the GGP50K from the American company Neogen, or the Zhongxin No. 1.

The present disclosure provides a non-naturally occurring 50K mSNP liquid-phase chip for pigs based on multiple single nucleotide polymorphisms (mSNPs), comprising the probe hybridization solution, wherein the set of DNA molecules (i.e., probes) is dissolved in a buffer solution and diluted to a concentration of 1-5 pmol/mL to form the probe hybridization solution.

In some embodiments, the buffer solution is a mixture of EDTA and Tris-HCl.

In some embodiments, the probe hybridization solution further includes:

| Component name | Quantity |
|---|---|
| Pooled, barcoded library | 0.6 μL |
| GenoBaits Block I | 5 μL |
| GenoBaits Block II | 2 μL |
| forILM/MGI | |
| The DNA molecules | 300 ng |

In some embodiments, the probe hybridization solution is concentrated to dryness using a vacuum concentrator at a temperature≤60° C.

The present disclosure provides an application of the 50K mSNP liquid-phase chip for pigs, specifically a screening method for pig breeding. The method includes the following steps: obtaining samples from the pigs to be tested and extracting genomic DNA constructing pig cDNA libraries; hybridizing and sequencing the constructed libraries with the liquid-phase chip; performing mSNP genotyping according to the sequencing data operation process, and determining the genotypes of all liquid-phase chip marker loci for each individual.

In some embodiments, the mSNP genotyping comprises:
Step 1: After determining the genotypes of all mSNP markers for the individual liquid-phase chip, perform quality control on the mSNP genotypes; the quality control is carried out in the following order:
1) Filter out multi-allelic variants;
2) Remove sex chromosomes and loci with unknown positions;
3) Remove SNPs with a call rate below 90%;
4) Remove SNPs with a minor allele frequency (MAF) below 0.05;
5) Remove individuals with a call rate below 90%.
Step 2: Using the target SNP loci as the core, define a 200 bp upstream and downstream region as a haplotype block, dividing the genome into 52,000 haplotype blocks, each with at least one mSNP marker, with varying numbers.
Step 3: For each haplotype block, infer haplotypes, determine haplotype alleles, and construct haplotype genotypes or diplotypes for each haplotype block in the tested sample, thereby constructing diplotype vectors for all haplotype blocks in the tested sample, similar to genotype vectors for all mSNP markers.
Step 4: Based on the diplotype vectors of all samples, apply genetic analysis or molecular breeding methods, with each haplotype block treated as a marker, and haplotypes within the block as alleles and diplotypes as genotypes.

Compared to existing technology, the method provided by the present disclosure for developing the 50K mSNP liquid-phase chip for pigs has the following beneficial effects.

The present disclosure provides a high-throughput 50K mSNP liquid-phase chip for pigs based on targeted capture sequencing for genotyping. The DNA molecules design considers the distribution of captured SNP loci across the genome, locus polymorphism, mSNP marker quality, and other issues. In the Duroc, Landrace, and Large White pig populations, the target loci MAF requirement is greater than 0.35, effectively avoiding issues such as uneven marker density and poor polymorphism.

Compared to existing liquid-phase chips, the present disclosure considers the quality of mSNP markers and the issue of linkage disequilibrium among mSNP markers. While adhering to the basic principles of liquid-phase chip design, genomic regions with moderate linkage disequilibrium between markers were selected, generating more SNP markers with high genotyping quality and moderate linkage disequilibrium within the DNA molecules region with the target loci. These markers are collectively referred to as mSNP. The mSNP liquid-phase chip can generate multiple SNP markers at a single amplification loci (target loci), expanding the number of detectable SNPs to 1.5-2 times that of the loci. This solves the problem of the relatively small number of high-quality mSNP markers in traditional liquid-phase chips without increasing costs.

Additionally, the present disclosure provides an effective method for utilizing mSNP markers. Compared to traditional single-marker analysis and haplotype analysis methods, the present disclosure provides a haplotype block method centered on the target loci that includes its upstream and downstream mSNPs. This method fully utilizes the linkage disequilibrium information of SNP markers within the DNA molecules, improving the efficiency of genetic analysis and molecular breeding. It avoids the noise caused by mSNPs within the DNA molecules in traditional single-marker analysis, which can result in excessive bias, as well as the low efficiency of fixed SNP number or fragment length haplotype analysis methods.

Therefore, based on the developed 50K liquid-phase chip for pigs and the characteristics of the pig genome, the present disclosure has developed the 50K mSNP liquid-phase chip for pigs using multiple single nucleotide polymorphism technologies. Compared to existing liquid-phase chips, the present disclosure increases the number of effective mSNP markers without increasing costs, resulting in greater information content. Concurrently, mSNPs can be leveraged in conjunction with haplotype and other analytical techniques to enhance the efficacy of genetic analysis and molecular breeding endeavors. Furthermore, the liquid-phase chip of the present invention can achieve DNA hybridization capture time within 1 hour, significantly shortening the time required to obtain genotypes compared to the overnight hybridization capture process that takes more than 16 hours. The entire process of library construction and capture can be completed within one day.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic diagram of solid-phase chip and liquid-phase chip technologies;

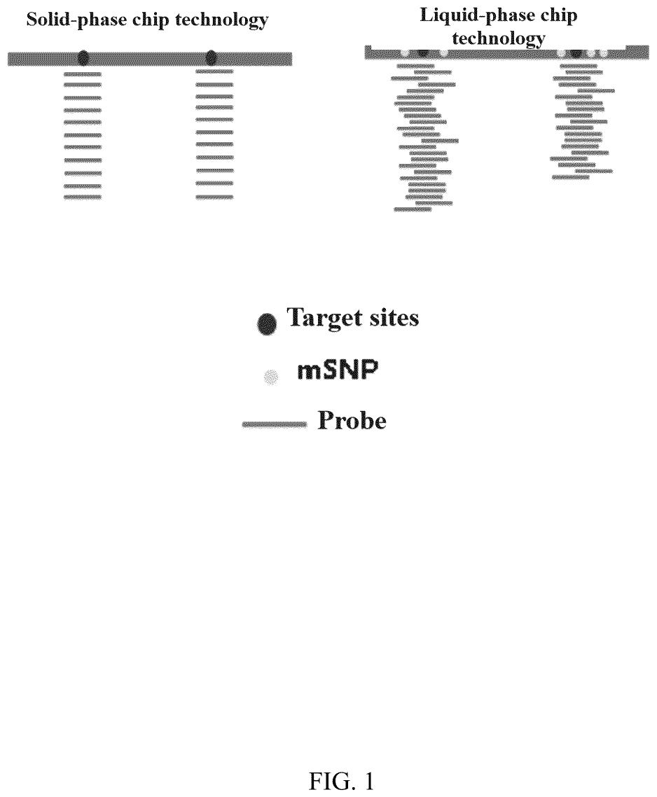

FIG. 2 illustrates the decay of linkage disequilibrium (LD) among five subspecies of rice (Yan et al., 2020); the five subspecies are Australian rice (Aus), Fragrant rice (Aro), Tropical rice, Japonica rice (TrJ), Temperate Japonica rice (TeJ), and the wild rice subspecies (Oru), as well as Indica rice. The markers among these five subspecies of rice indicate that the R² value for intervals of several hundred base pairs is 1, which signifies complete LD.

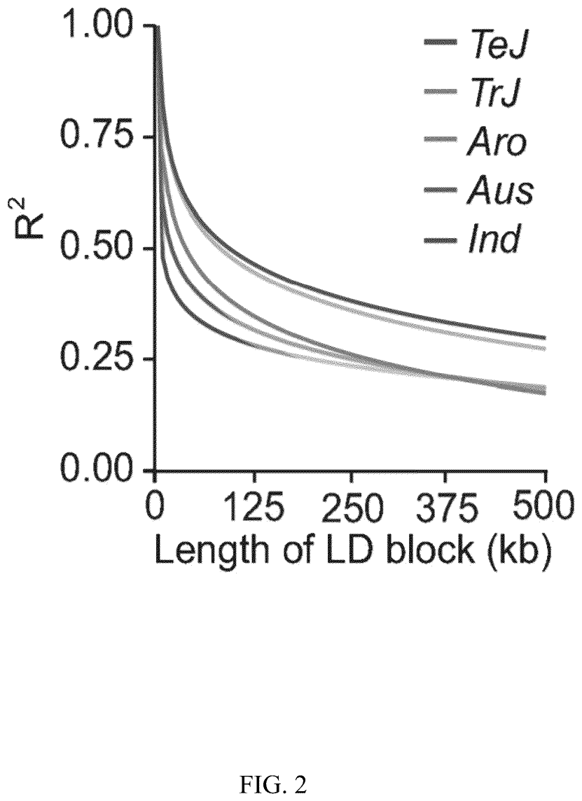

FIG. 3 shows the number (A) and density distribution (B) of target SNP loci on each chromosome in the present disclosure;

FIG. 4 shows the distribution of target loci (in red) and mSNPs (in blue) on each chromosome for the 50K mSNP liquid-phase chip in Farm 1;

FIG. 5 shows the density distribution of target loci (A) and all mSNPs (B) on each chromosome for the 50K mSNP liquid-phase chip in Farm 1;

FIG. 6 shows the distribution (A) and decay of linkage disequilibrium (B) of all loci after quality control for the 50K mSNP liquid-phase chip;

FIG. 7 shows the process of constructing a haplotype matrix; and

FIG. 8 shows the distribution of the number of mSNPs for each DNA molecules (A) and the average linkage disequilibrium level between adjacent markers under single-marker and different haplotype block division schemes (B).

### DETAILED DESCRIPTION

Below, the specific embodiments of the present disclosure are described in further detail in conjunction with the accompanying drawings and examples. The following examples are intended to illustrate the present disclosure but are not intended to limit its scope.

### Example 1: Screening Method for SNP Markers and Probe Preparation

### Step 1: Target SNP Marker Selection

In conjunction with the 50K liquid-phase chip invented by the present disclosure, based on the whole-genome sequencing data of the Duroc, Large White, and Landrace pig breeds, comparison is made to the pig reference genome Sscrofa11.1. Genomic regions with a moderate degree of linkage disequilibrium between markers are selected, and the target SNP loci from the genomic regions are screened. The principles for screening target SNP loci are: (1) uniform distribution across chromosomes, with denser distribution at both ends of the chromosomes; (2) polymorphism mainly considers MAF>0.35 in Duroc, Landrace, and Large White pigs; (3) the average linkage disequilibrium level (r²) with upstream and downstream SNP markers is less than 0.85; (4) comparing with the QTLdb database, aiming for SNP markers to be located in QTL regions related to economic traits; (5) there is partial overlap with some loci of the 50K chips currently on the market (50K SNP liquid-phase chip, Neogen's GGP50K in the United States, and Zhongxin No. 1), especially including the important candidate loci for growth, reproduction, feed conversion, and body size traits that the applicant had previously developed for the 50K SNP liquid-phase chip (CN202110359470.7-A 50K liquid-phase chip for pigs based on targeted capture sequencing and its application), ensuring chip compatibility.

For downstream functional interpretation of the detected variants, previously reported quantitative trait loci (QTL) were retrieved from the Animal QTL Database (Animal QTLdb). The Animal QTLdb is an online, curated repository that collects and standardises published QTL and association results for livestock species, including pigs, and provides genome-based tools to compare, confirm and locate QTL on the corresponding reference genome. In particular, the pig-specific sub-database (Pig QTLdb) can be accessed via https://www.animalgenome.org/cgi-bin/QTLdb/SS/index. Across the full SNP panel (108,533 loci), 8,641 loci (7.96%) lie within the QTL regions (42,163 regions in total). Overlap is observed on all autosomes (Chr 1-18), with an uneven distribution by chromosome: Chr 3 contains the highest number of overlapping SNPs (1,020 loci), whereas Chr 18 contains the fewest (131 loci). The per-chromosome counts show a central tendency of 444.5 overlapping SNPs (median; interquartile range 309-616), with a mean of 480.1 loci per chromosome. These results indicate that a substantial subset of markers reside in QTL-enriched regions, providing dense coverage over trait-associated intervals while preserving chromosomal specificity. The important 20 SNPs are shown in Table 1.

TABLE 1
20 important SNPs paired with QTL

| | | | | QTL_ | QTL_ |
|---|---|---|---|---|---|
| ID | CHR | BP | QTL | START_BP | END_BP |
| 1_373626 | 1 | 373626 | Loin weight | 373624 | 373628 |
| | | | QTL (153573) | | |
| 1_381075 | 1 | 381075 | Backfat at rump | 381073 | 381077 |
| | | | QTL (23331) | | |
| 1_381075 | 1 | 381075 | Average daily | 381073 | 381077 |
| | | | gain QTL | | |
| | | | (28795) | | |
| 2_44048 | 2 | 44048 | Fat | 44046 | 44050 |
| | | | androstenone | | |
| | | | level QTL | | |
| | | | (194766) | | |
| 3_537968 | 3 | 537968 | Intramuscular | 537966 | 537970 |
| | | | fat content QTL | | |
| | | | (176598) | | |
| 4_102268 | 4 | 102268 | Number of | 102266 | 102270 |
| | | | mummified pigs | | |
| | | | QTL (178848) | | |
| 5_2378170 | 5 | 2378170 | Gestation length | 2378168 | 2378172 |
| | | | QTL (263458) | | |
| 6_128223 | 6 | 128223 | Front leg | 128221 | 128225 |
| | | | conformation | | |
| | | | QTL (16465) | | |
| 7_651731 | 7 | 651731 | Lean meat | 651729 | 651733 |
| | | | percentage QTL | | |
| | | | (216186) | | |
| 8_1195035 | 8 | 1195035 | Days to 115 kg | 1195033 | 1195037 |
| | | | QTL (130571) | | |
| 9_2471426 | 9 | 2471426 | Shear force QTL | 2471424 | 2471428 |
| | | | (22436) | | |
| 10_951741 | 10 | 951741 | LDL cholesterol | 951739 | 951743 |
| | | | QTL (55990) | | |
| 11_115523 | 11 | 115523 | indole, | 115521 | 115525 |
| | | | laboratory | | |
| | | | QTL (18659) | | |
| 12_94345 | 12 | 94345 | Palmitic acid to | 94343 | 94347 |
| | | | myristic | | |
| | | | acid ratio | | |
| | | | QTL (101429) | | |
| 13_557266 | 13 | 557266 | Interleukin-10 | 557264 | 557268 |
| | | | level QTL | | |
| | | | (23461) | | |
| 14_962165 | 14 | 962165 | Litter weight, | 962163 | 962167 |
| | | | total QTL | | |
| | | | (179299) | | |
| 15_769750 | 15 | 769750 | Interferon- | 769748 | 769752 |
| | | | gamma to | | |
| | | | interleukin-10 | | |
| | | | ratio QTL | | |
| | | | (23472) | | |
| 16_933181 | 16 | 933181 | Coping behavior | 933179 | 933183 |
| | | | QTL (124304) | | |
| 17_693319 | 17 | 693319 | Number of visits | 693317 | 693321 |
| | | | to feeder QTL | | |
| | | | (22403) | | |
| 18_1102523 | 18 | 1102523 | Feed conversion | 1102521 | 1102525 |
| | | | ratio QTL | | |
| | | | (62277) | | |

### Step 2: Design Probes (DNA Molecules) Based on the Target SNIP Loci

DNA molecules are optimized and designed using multiple single nucleotide-polymorphism (mSNP). For each of the target SNP loci, 1-4 DNA molecules of 110 bp length are designed, with each of the DNA molecules covering the target SNP loci. The total coverage of DNA molecules centered on the target SNP loci is 165 bp in length. The principles of DNA molecules design are: 1) Select DNA molecules with GC content between 30%-80%; 2) Select regions with a homology number ≤5; 3) Exclude regions containing SSR or N regions in the DNA molecules. GC content refers to the total proportion of guanine (G) and cytosine (C) bases in the DNA molecules. When designing and screening the DNA molecules, selecting only those with GC content between 30%-80% ensures that the designed DNA molecules exhibit stable hybridization, high specificity, and excellent uniformity.

Step 3: Through Multiple Sequencing and Hybridization of Probes, Select Probes Containing High-Quality mSNPs, and Optimize the Probes.

Probes are sequenced and hybridized to detect the genotyping quality of target SNP loci and mSNPs. Set a missing rate of NA<0.1, a minimum allele frequency (MAF)≥0.05, and heterozygosity (Het)<0.5 as standards to screen mSNPs, with removal of those that do not meet the standards. If the probe does not meet the genotype quality control requirements of mSNPs, delete the probe and the corresponding target SNP loci, redesign new probes according to Steps 1 and 2, and continue to test and optimize the probes as per this step. The mSNPs that meet the quality inspection requirements are finally used as 50K mSNP liquid-phase chip loci.

The method described in step three is designed to ensure the optimal quantity and quality of mSNP markers (including target SNP loci) under the same target SNP probe. The present disclosure includes 52,000 target loci and ultimately 80,631 high-quality probes (DNA molecules). The sequence information of the probes (DNA molecules), including SEQ ID NOs: 1-80,631, has been provided in the sequence listing herein provided. The probes containing 5-7 mSNPs have an average interval of 550 Kb. Table 2 lists some of the probe information containing 5-7 mSNPs on chromosome 18.

TABLE 2
Information on probes containing 5-7 mSNP markers (chromosome 18)

| | | | | | Number | | |
|---|---|---|---|---|---|---|---|
| | | | | | of | | |
| | | | | Probe | mSNP | | |
| | | Target | Probe | End | Markers | | |
| | | Loci | Start | Posi- | in | | SNP |
| Probe | | Position | Position | tion | the | Detectable | Se- |
| ID | Probe Sequence | (bp) | (bp) | (bp) | Probe | SNP ID | quence |
| 18- | TATTTGCAGAGTCCC | 1432455 | 1432373 | 1432482 | 5 | 18_1432405; | G/A; |
| 1432455 | AGCTGCCCCATCTAG | | | | | 18_1432414; | C/T; |
| | TGATCTCTGCATGGA | | | | | 18_1432438; | G/A; |
| | GCCACCCGGCAGGCC | | | | | 18_1432455; | G/T; |
| | CTGGTAACCAGGTCG | | | | | 18_1432470 | A/T |
| | AGACACATTTTCCTT | | | | | | |
| | TGTCCCATTGTCCAA | | | | | | |
| | ACTGG | | | | | | |
| | (SEQ ID NO: | | | | | | |
| | 30,687) | | | | | | |
| 18- | TCCCAAGTCCCTGAT | 2119817 | 2119764 | 2119873 | 5 | 18_2119817; | C/T; |
| 2119817 | CCTGCAGTGGTCCTC | | | | | 18_2119826; | T/C; |
| | TGAGCACGGGGACAG | | | | | 18_2119845; | C/G; |
| | AAAACACACGCGCTT | | | | | 18_2119847; | C/T; |
| | TGCGGGGCCCTGACT | | | | | 18_2119872 | T/C |
| | CCCTTGGGTTTGACG | | | | | | |
| | TAAGGGTGGTTCAGT | | | | | | |
| | AACCC | | | | | | |
| | (SEQ ID NO: | | | | | | |
| | 30,697) | | | | | | |
| 18- | ATATAAAGAGTTTCC | 2252278 | 2252251 | 2252360 | 5 | 18_2252278; | 0/C; |
| 2252278 | TTGGTTTTCATGCTG | | | | | 18_2252281; | C/G; |
| | GCAGTGCCAGGGCAC | | | | | 18_2252325; | A/G; |
| | CAAATCCCTCAGAGC | | | | | 18_2252340; | C/T; |
| | TCGTCAACCAGCCCG | | | | | 18_2252341 | A/G |
| | GCTGCACTCCACGCT | | | | | | |
| | GGCTGTGACTTTACA | | | | | | |
| | GATAG | | | | | | |
| | (SEQ ID NO: | | | | | | |
| | 30,702) | | | | | | |
| 18- | CCAGCCCTGCCCACA | 2514413 | 2514331 | 2514440 | 6 | 18_2514345; | A/G; |
| 2514413 | CCCAGGTCTTAGATG | | | | | 18_2514349; | A/G; |
| | TCCAGCTTCCAGGAC | | | | | 18_2514367; | T/C; |
| | TGAGAGAGACTCTGT | | | | | 18_2514376; | C/T; |
| | TTCTGCTGCTTAAGC | | | | | 18_2514396; | C/T; |
| | TGCCCTGGGAAACTA | | | | | 18_2514413 | A/G |
| | ACGCAGAACAAGTAA | | | | | | |
| | ATAAA | | | | | | |
| | (SEQ ID NO: | | | | | | |
| | 30,711) | | | | | | |
| 18- | GAAACCAGGCCTGCT | 2724065 | 2724038 | 2724147 | 6 | 18_2724065; | T/C; |
| 2724065 | CGCCCCCACGGTTAA | | | | | 18_2724123; | A/C; |
| | GGCTACTCGGCTTTG | | | | | 18_2724125; | C/T; |
| | AGACAACCAGGCTGA | | | | | 18_2724131; | G/A; |
| | AATCACCTGTGTTTT | | | | | 18_2724134; | A/C; |
| | GTTGGTGCTCCGTCT | | | | | 18_2724145 | A/T |
| | GCCAAGCAGCGAAAG | | | | | | |
| | CCTTC | | | | | | |
| | (SEQ ID NO: | | | | | | |
| | 30,722) | | | | | | |
| 18- | TATATGGCAACCAAA | 2816068 | 2816015 | 2816124 | 7 | 18_2816034; | C/T; |
| 2816068 | AACATGGCAGGGCGA | | | | | 18_2816047; | A/G; |
| | TGAATGGGAGTGGGT | | | | | 18_2816052; | A/G; |
| | GGTCGTTACAGCTGG | | | | | 18_2816068; | T/C; |
| | TGATGAGCGTATTTT | | | | | 18_2816077; | G/A; |
| | AGTTCATTGTTCTAG | | | | | 18_2816080; | G/A; |
| | TCTCTGGACTTTGGT | | | | | 18_2816090 | G/A |
| | GTCAG | | | | | | |
| | (SEQ ID NO: | | | | | | |
| | 30,725) | | | | | | |
| 18- | CATCAGAGAAAGGAG | 3594827 | 3594774 | 3594883 | 6 | 18_3594782; | G/A; |
| 3594827 | ATTAAAATGACAATG | | | | | 18_3594827; | O/T; |
| | AGATCCCATTACCCA | | | | | 18_3594854; | G/C; |
| | CCCACCAATCTGTCA | | | | | 18_3594876; | C/G; |
| | AAAATGGGGGAGGGG | | | | | 18_3594878; | A/G; |
| | AGCTGCTGGCCTCCA | | | | | 18_3594880 | A/G |
| | CACTGCTGATGCGCG | | | | | | |
| | TGTAA | | | | | | |
| | (SEQ ID NO: | | | | | | |
| | 30,764) | | | | | | |
| 18- | CCTTGTCGCTGAAGG | 3817775 | 3817748 | 3817857 | 7 | 18_3817754; | T/C; |
| 3817775 | GCAACGCCACTGTTT | | | | | 18_3817775; | C/T; |
| | CTCTGACTCTCTCTG | | | | | 18_3817783; | C/A; |
| | CAGCCAACTGGTGGT | | | | | 18_3817829; | G/T; |
| | GGGAGCTGCACAGAG | | | | | 18_3817836; | C/T; |
| | GCTTGTTTACTGCTG | | | | | 18_3817845; | G/A; |
| | GGGGCAGAGGGGGAT | | | | | 18_3817847 | A/G |
| | GCAGA | | | | | | |
| | (SEQ ID NO: | | | | | | |
| | 30,773) | | | | | | |
| 18- | ACAGTCATTTTGGTT | 3864387 | 3864360 | 3864469 | 5 | 18_3864387; | C/T; |
| 3864387 | TCTCCTTGAGCCTGG | | | | | 18_3864393; | G/C; |
| | CTCGTCGGGATGGTG | | | | | 18_3864425; | 0/C; |
| | AGTCTGGAAGGCACC | | | | | 18_3864435; | G/A; |
| | CAGACCCCATGGCTG | | | | | 18_3864452 | T/C |
| | AGGGCGGAGGGCATG | | | | | | |
| | CACGGAGTTGGGTCT | | | | | | |
| | TTGAA | | | | | | |
| | (SEQ ID NO: | | | | | | |
| | 30,775) | | | | | | |
| 18- | AGGCAGTGCCCCTCT | 3963219 | 3963166 | 3963275 | 6 | 18_3963174; | A/C; |
| 3963219 | TAGTAAAGATGACAC | | | | | 18_3963181; | C/T; |
| | CTAAAGGTGCTTCCC | | | | | 18_3963192; | C/A; |
| | TGAGTCCAAGCAGGT | | | | | 18_3963199; | G/A; |
| | GATGTGCTGGGTGAC | | | | | 18_3963219; | A/G; |
| | TGAGAAGCTGGGGTA | | | | | 18_3963225 | C/T |
| | TTAACCACCATCTAT | | | | | | |
| | TTTTC | | | | | | |
| | (SEQ ID NO: | | | | | | |
| | 30,782) | | | | | | |
| 18- | TAAGATGAATGACCC | 3988979 | 3988897 | 3989006 | 6 | 18_3988902; | T/C; |
| 3988979 | AGGGGTCAGTGCCGA | | | | | 18_3988950; | G/A; |
| | ATTAGGGAAGGATAA | | | | | 18_3988979; | A/G; |
| | ACCCTTCCGTGCCTC | | | | | 18_3988985; | T/C; |
| | ATCCTCTTCCCTGTA | | | | | 18_3988989; | A/G; |
| | CACCCAGAGTCCGTG | | | | | 18_3988990 | T/C |
| | GCATTCGGATGAGGA | | | | | | |
| | AGTCC | | | | | | |
| | (SEQ ID NO: | | | | | | |
| | 30,783) | | | | | | |
| 18- | CACAGGCTGATGCCC | 4317527 | 4317474 | 4317583 | 6 | 18_4317488; | T/C; |
| 4317527 | ACACGAGGGTCTCAA | | | | | 18_4317502; | G/A; |
| | TGGGCCATGGGAACA | | | | | 18_4317514; | A/G; |
| | GATGCAATGCCGTGC | | | | | 18_4317527; | A/G; |
| | AAACATTTCCAGCTG | | | | | 18_4317528; | A/C; |
| | GGTTGTTGGCAGCCC | | | | | 18_4317582 | T/C |
| | GTGACTCAGGGTCCC | | | | | | |
| | CATCT | | | | | | |
| | (SEQ ID NO: | | | | | | |
| | 30,799) | | | | | | |
| 18- | TGTCTGGCACTTTCC | 4606302 | 4606275 | 4606384 | 5 | 18_4606302; | A/G; |
| 4606302 | TTCTCCCAGGGCGGC | | | | | 18_4606331; | G/A; |
| | TGCGGGCAGGATCAG | | | | | 18_4606356; | G/A; |
| | AGCTTCGAGGCAGCC | | | | | 18_4606360; | G/A; |
| | ATTCTGGGCTCTTGT | | | | | 18_4606380 | A/G |
| | TGCATCATTTATCAT | | | | | | |
| | GAAAACGAGGCATTC | | | | | | |
| | GAATT | | | | | | |
| | (SEQ ID NO: | | | | | | |
| | 30,823) | | | | | | |
| 18- | CCTGGGAACCTCCGT | 5534441 | 5534359 | 5534468 | 6 | 18_5534382; | C/T; |
| 5534441 | ATGTCGCACCTGTGG | | | | | 18_5534387; | G/A; |
| | CCCTGAAAGAAAAAC | | | | | 18_5534416; | G/A; |
| | AAACATACAAACGAT | | | | | 18_5534422; | G/A; |
| | GAAGTCAGCGTGACA | | | | | 18_5534428; | G/C; |
| | TCACCCATCTCTGAC | | | | | 18_5534441 | T/G |
| | ACCGGAAGTACTCTA | | | | | | |
| | GGGTT | | | | | | |
| | (SEQ ID NO: | | | | | | |
| | 30,855) | | | | | | |
| 18- | ATGAGGGGCCAGAGG | 5630257 | 5630230 | 5630339 | 7 | 18_5630232; | G/T; |
| 5630257 | AAGGGCTGGCAGCCT | | | | | 18_5630255; | 0/A; |
| | GATCGCACACGGAGC | | | | | 18_5630257; | C/T; |
| | AGCTGGGCTCGCAAA | | | | | 18_5630280; | G/A; |
| | ATCCAAGCTCCTCAA | | | | | 18_5630286; | 0/C; |
| | GGTCTGCCTGGGCCG | | | | | 18_5630319; | T/G; |
| | CTTCTCCCTTGCCCA | | | | | 18_5630337 | 0/G |
| | CCGTT | | | | | | |
| | (SEQ ID NO: | | | | | | |
| | 30,862) | | | | | | |
| 18- | AGCTGTCCTCCTGCC | 6301770 | 6301688 | 6301797 | 6 | 18_6301693; | C/T; |
| 6301770 | ATACTCTATCTTCCA | | | | | 18_6301711; | A/T; |
| | CATGGTACTCAGATG | | | | | 18_6301770; | A/G; |
| | TGATGGCTGGAGCTC | | | | | 18_6301774; | A/C; |
| | CAGCAGTCACTTTGG | | | | | 18_6301775; | G/A; |
| | ACTAGGAAGCCCAGG | | | | | 18_6301796 | A/G |
| | TCCATATCCTAGGTG | | | | | | |
| | GCTGC | | | | | | |
| | (SEQ ID NO: | | | | | | |
| | 30,887) | | | | | | |
| 18- | GCCCCTCATTTGCTG | 7110407 | 7110325 | 7110434 | 6 | 18_7110356; | T/C; |
| 7110407 | TGGGTCTTAGGGCCC | | | | | 18_7110368; | T/C; |
| | CCGCTTTCCCTTTCG | | | | | 18_7110395; | G/A; |
| | GCGAGAACGGCCCCT | | | | | 18_7110398; | G/C; |
| | CCCTCCTCTGAGTCT | | | | | 18_7110407; | G/A; |
| | TTGTCTCACCCTCTT | | | | | 18_7110430 | A/C |
| | CATGGACAAGGAGAA | | | | | | |
| | CCCAT | | | | | | |
| | (SEQ ID NO: | | | | | | |
| | 30,910) | | | | | | |
| 18- | CCCCTCCCTCCTCTG | 7110407 | 7110380 | 7110489 | 5 | 18_7110395; | G/A; |
| 7110407 | AGTCTTTGTCTCACC | | | | | 18_7110398; | G/C; |
| | CTCTTCATGGACAAG | | | | | 18_7110407; | G/A; |
| | GAGAACCCATACCCT | | | | | 18_7110430; | A/C; |
| | CTCCTCAGGAAAGCT | | | | | 18_7110451 | C/A |
| | TCTTGGAGAGAACAC | | | | | | |
| | AGCTCTAACATTTCT | | | | | | |
| | GGATC | | | | | | |
| | (SEQ ID NO: | | | | | | |
| | 30,911) | | | | | | |
| 18- | AGAGATTGGCTATGC | 7634894 | 7634812 | 7634921 | 5 | 18_7634851; | T/C; |
| 7634894 | CTGGGGTTTTAGGCA | | | | | 18_7634866; | A/T; |
| | TAAACTAAGCAACAG | | | | | 18_7634867; | G/A; |
| | CCCTGCAGATATTAG | | | | | 18_7634894; | C/A; |
| | CATCTTGATTTGTTC | | | | | 18_7634915 | A/C |
| | AAGGAATACTCCTGG | | | | | | |
| | AACCATAGCTAGGCG | | | | | | |
| | CAGGC | | | | | | |
| | (SEQ ID NO: | | | | | | |
| | 30,932) | | | | | | |
| 18- | ATTAGCATCTTGATT | 7634894 | 7634867 | 7634976 | 5 | 18_7634867; | G/A; |
| 7634894 | TGTTCAAGGAATACT | | | | | 18_7634894; | C/A; |
| | CCTGGAACCATAGCT | | | | | 18_7634915; | A/C; |
| | AGGCGCAGGCACAAA | | | | | 18_7634929; | G/T; |
| | GCTTTGACATGTTCA | | | | | 18_7634947 | G/A |
| | CCCCCAGAATTCTAT | | | | | | |
| | TGGGGTAAAGAAGGA | | | | | | |
| | GAGTG | | | | | | |
| | (SEQ ID NO: | | | | | | |
| | 30,933) | | | | | | |
| 18- | TAAGAGCTTACAATT | 7742000 | 7741973 | 7742082 | 5 | 18_7741995; | A/G; |
| 7742000 | GTTACACGCTTTTGA | | | | | 18_7742000; | G/T; |
| | CTAATCAGGCATTGG | | | | | 18_7742028; | A/G; |
| | TCCAAGTTCTGCAGA | | | | | 18_7742030; | G/A; |
| | TGGTAAACCCCTCCG | | | | | 18_7742052 | A/G |
| | CATCGGCACGAGGCA | | | | | | |
| | GATGATGATTAGCCC | | | | | | |
| | ATTCT | | | | | | |
| | (SEQ ID NO: | | | | | | |
| | 30,938) | | | | | | |
| 18- | TGCATGGCTTCACAG | 8245730 | 8245648 | 8245757 | 6 | 18_8245650; | C/T; |
| 8245730 | TTCTGGAGTCTAGAA | | | | | 18_8245710; | A/G; |
| | GTCTAAAACCAAGGT | | | | | 18_8245716; | C/G; |
| | GTAAGCAGGGCTCTG | | | | | 18_8245730; | T/C; |
| | AGAGAGAACCTGTTC | | | | | 18_8245735; | A/G; |
| | CTTGCTTCTGGCAGT | | | | | 18_8245736 | G/A |
| | CTTTGCTGTTCTTGG | | | | | | |
| | CTTGT | | | | | | |
| | (SEQ ID NO: | | | | | | |
| | 30,959) | | | | | | |
| 18- | CTCTGAGAGAGAACC | 8245730 | 8245703 | 8245812 | 6 | 18_8245710; | A/G; |
| 8245730 | TGTTCCTTGCTTCTG | | | | | 18_8245716; | C/G; |
| | GCAGTCTTTGCTGTT | | | | | 18_8245730; | T/C; |
| | CTTGGCTTGTGGATG | | | | | 18_8245735; | A/G; |
| | TATCTCTGATCTCTG | | | | | 188245736; | G/A; |
| | CTGCCTTCACTCTCA | | | | | 18_8245796 | A/G |
| | CACGGTGTTAGCCTG | | | | | | |
| | TGTCT | | | | | | |
| | (SEQ ID NO: | | | | | | |
| | 30,960) | | | | | | |

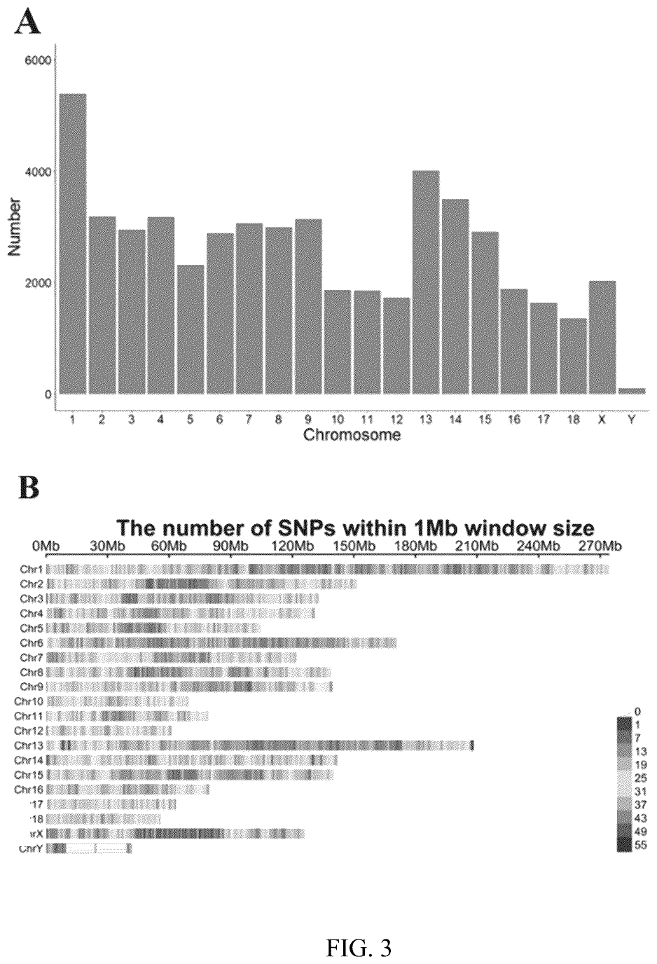

Compared to the 50K SNP solid-phase chip on the market, the number of target SNP loci provided by the present disclosure is 52,000 (FIG. 3). A total of 80,631 probes were designed for the target loci, and the number of detected SNP markers (mSNP, including target loci) was significantly increased to 80,000-100,000, providing more genomic information.

### Example 2: 50K mSNP Liquid-Phase Chip Preparation

This example demonstrates the preparation process of the 50K mSNP liquid-phase chip of the present invention. The specific steps are as follows:
Step 1: Probe Preparation
Mix the synthesized DNA molecules in equimolar amounts. Use EDTA and Tris-HCl (TE buffer) to dissolve to 3 pmol/mL, and prepare a 50K probe hybridization solution for subsequent sequencing and hybridization.
1. Preparation of TE Buffer
1×TE Buffer
Component concentration: 10 mM Tris-HCl, 1 mM EDTA, pH=8.0
Preparation volume: 500 mL
Preparation method: Measure the following solutions into a 500 mL beaker:
1M Tris-HCl Buffer, pH=8.0, 5 ml; 0.5 M EDTA, pH=8.0, 1 ml
Add about 400 ml dd H₂O to the beaker, mix well; then dilute the solution to 500 ml, and sterilize at high temperature and pressure; store at room temperature.
2. Use the prepared TE buffer to dissolve the probes and prepare a 3 pmol/mL 50K probe hybridization solution for subsequent sequencing and hybridization.
Step 2: Preparation of Probe Hybridization Solution
1. According to the library type, mix the following reagents in a 1.5 mL PCR tube:

| Component name | Quantity |
|---|---|
| Pooled, barcoded library | 0.6 μL |
| GenoBaits Block I | 5 μL |
| GenoBaits Block II | 2 μL |
| forILM/MGI | |
| DNA molecules | 300 ng |

2. Use a vacuum concentrator at a temperature of ≤60° to concentrate to dryness;
3. After concentration, centrifuge at 12,000 rpm for 1 min. The prepared probes can be stored overnight at room temperature (15-25° C.) for subsequent DNA library hybridization capture.

### Example 3: Use and Detection Method of the 50K Liquid-Phase Chip

This example demonstrates the operational process for using the 50K liquid-phase chip of the invention for genotyping. The specific steps are as follows:
Step 1: Obtaining and extracting genomic DNA from the pig sample;
Select three common commercial pig breeds: Duroc, Large White, and Landrace, with 20 samples from each breed, totaling 60 samples. Extract genomic DNA from ear tissue. The specific method is as follows:
1. Shred the appropriate amount of ethanol-dehydrated pig ear tissue and place it in a 96-well deep plate (use a 2.0 mL centrifuge tube if the amount is small). Add a 5 mm steel bead, freeze with liquid nitrogen, and grind with a grinder for 1-2 minutes.
2. Add 500 μL Buffer PL2 and 5 μL Proteinase K (the diluted Proteinase K currently used in the lab) to the deep-well plate. Secure the cap and mix well using a shaker.
3. Incubate at 65° C. for 30 min, periodically invert the plate for mixing during incubation.
4. Add 500 μL phenol-chloroform-isoamyl alcohol to the deep-well plate, mix well by shaking or pipetting up and down, and let it stand for 5 min.
5. Centrifuge at 4000 rpm for 10 min, transfer 400 μL of the supernatant to a new 96-well deep-well plate. (Ensure not to aspirate the middle sediment).
6. Add 800 μL PW solution and mix well.
7. Transfer the supernatant from step 6 to a 96-well centrifuge column in two batches, and perform vacuum filtration.
8. Add 600 μL WB I to the 96-well centrifuge column, incubate at room temperature for 2 min, and perform vacuum filtration. (Ensure anhydrous ethanol has been added to WB I as specified on the bottle).
9. Add 600 μL WB II to the 96-well centrifuge column and perform vacuum filtration. (Ensure anhydrous ethanol has been added to WB II as specified on the bottle).
10. Add 600 μL WB II to the 96-well centrifuge column and perform vacuum filtration.
11. Place the 96-well centrifuge column into an empty collection plate, centrifuge at 4000 rpm for 5 min. Place the 96-well centrifuge column on a new 96-well PCR plate and air dry at room temperature.
12. Add 60-100 μL preheated 65° C. TE to the 96-well centrifuge column, incubate at room temperature for 2 min, and centrifuge at 4000 rpm for 5 min (preheating the TE to 65° C. helps improve DNA elution efficiency and gel electrophoresis detection of target fragment length).
Step 2: Constructing a pig cDNA library;
The specific steps include:
1. Probe Mixture
a) In a PCR tube, prepare the following reaction using the reagents of the present invention:

| DNA (1 ng-200 ng) | _ | μL |
|---|---|---|
| Nuclease-free water | _ | μL |
| GenoBaits End Repair Buffer | 4 | μL |
| GenoBaits End Repair Enzyme | 3.1 | μL |
| Total | 20 | L |

b) After gently mixing the reaction system, briefly centrifuge to collect the reaction liquid at the bottom of the tube.
c) Place the reaction tube in a PCR instrument for the following reaction at 82° C. with the hot lid on:

| | | |
|---|---|---|
| 37° C. | 20 | min |
| 72° C. | 20 | min |
| Hold at | 4° | C. |

2. Adaptor ligation
a) Directly add the following components to the reaction system from Step 1:

| | | |
|---|---|---|
| GenoBaitsULtra DNA Ligase | 2 | μL |
| GenoBaitsULtra DNA Ligase Buffer | 8 | μL |
| GenoBaits Adapter for MGI | 2 | μL |
| Nuclease-free water | 8 | μL |
| Total | 20 | L |

b) After gently mixing the reaction system, briefly centrifuge to collect the reaction liquid at the bottom of the tube.
Note: The system must be thoroughly mixed; otherwise, library construction may fail.
c) Place the reaction tube in a PCR instrument for the following reaction, and cancel the hot lid: incubate at 22° C. for 60 minutes, and then store at 4° C. for later use.
3. DNA Purification
a) Take out GenoPrep DNA Clean Beads in advance and equilibrate at room temperature for over 30 min; vortex to mix before use.
b) Add 48 μL GenoPrep DNA Clean Beads to the ligation system from step 2, mix by vortexing, avoiding bubbles as much as possible; let stand for 5 min, and then briefly centrifuge to collect the liquid at the bottom of the tube.
c) Place the tube on a magnetic rack for at least 3 min until the solution is clear; remove the supernatant.
d) Keep the PCR tube on the magnetic rack, add 100 μL of 80% ethanol. Incubate at room temperature for 30 seconds, remove the supernatant.
e) Keep the PCR tube on the magnetic rack, open the cap and air dry for 5 minutes until the ethanol evaporates completely.
f) Remove the PCR tube from the magnetic rack and resuspend the beads with the PCR system in Step 4.
4. Library Amplification
Starting Amount and Recommended Amplification Cycles

| Starting Amount | Recommended Amplification Cycles |
|---|---|
| 1 ng-10 ng | 8 |
| 10-100 ng | 6-8 |
| 100 ng and above | 6 |

a) Prepare the following reaction in a new tube:

| | | |
|---|---|---|
| GenoBaits PCR Master Mix | 10 | μL |
| I5 Barcode (10 μm)-MGI | 1 | μL |
| I7 Barcode (2 μm)-MGI | 5 | μL |
| Nuclease-free water | 4 | μL |
| Total | 20 | μL |

b) Add the above system to the beads dried in step 3, resuspend the beads, and briefly centrifuge to collect the reaction liquid at the bottom of the tube.
c) Place the reaction tube in the PCR instrument for the following reaction:

| | | | |
|---|---|---|---|
| 98° C. | 2 | min | |
| 98° C. | 30 | s | 6-8 cycles |
| 50° C. | 30 | s | |
| 72° C. | 40 | s | |
| 72° C. | 4 | min | |

5: Purification
a) Add 20 μL GenoPrep DNA Clean Beads to the system from step 4, mix by vortexing, avoiding bubbles as much as possible; let stand for 5 min, and then briefly centrifuge to collect the liquid at the bottom of the tube.
b) Place the tube on a magnetic rack for at least 3 min until the solution is clear; remove the supernatant.
c) Keep the PCR tube on the magnetic rack, add 100 μL of 80% ethanol. Incubate at room temperature for 30 seconds, remove the supernatant.
d) Keep the PCR tube on the magnetic rack, and air-dry with the cap open for 10 minutes.
e) Remove the PCR tube from the magnetic rack, add 35 μL Tris-HCl, vortex to mix, let stand for 5 min, and then briefly centrifuge to collect the liquid at the bottom of the tube.
f) On the magnetic rack, wait until the solution clears (about 3 minutes), and transfer the supernatant to a new tube, store at −20° C.
g) The library requires further quality testing (e.g., concentration measurement and distribution assessment) for subsequent sequencing or the next step of the experiment.
Step 3: Hybridization of pig genomic fragments with the probe of the present disclosure, PCR of the samples, and purification, followed by sequencing;
The steps are as follows:
1. Use the mixed probe of the present disclosure, melt at room temperature (15-25° C.), mix well, and briefly centrifuge.
2. GenoBaitsBlock II, GenoBaitsBlock
a) According to the library type, mix the following reagents in a 1.5 mL PCR tube:

| Component name | Quantity | |
|---|---|---|
| Pooled, barcoded Library | 0.6-1 | μg |
| GenoBaitsBlock I | 5 | μg (5 μL) |
| GenoBaitsBlock II for ILM/MGI | 2 | μL |
| Invention probe | 300 | ng |

b) Use a vacuum concentrator at a temperature of 560° C. to concentrate to dryness;
c) After concentration is complete, centrifuge at 12000 rpm for 1 min, and then proceed with subsequent operations.
3. Hybridization capture of the DNA library
a) Dissolve all GenoBaits hybridization reagents at room temperature;
b) Add the reagents to the tube;
c) Pipette or vortex to mix well, centrifuge at 12000 rpm for 1 min, let stand at room temperature for 5 min, pipette or vortex to mix again, lightly centrifuge, and transfer the entire mix to a 0.2 mL EP tube;
d) Thermal cycling incubation conditions: 95° C. for 10 min (lid temperature at 105° C.);
e) Once the PCR cycler cools down to 65° C., transfer it to another PCR machine with a lid temperature of 75° C. and 65° C. for hybridization. Note: If necessary, the experiment can be conducted overnight at 65° C. (14-16 h). *Hybridization at 65° C. helps improve capture efficiency.
4. Preparation of elution buffer (Wash Buffer)
Single capture system, dilute GenoBaits buffers to 1× system.
5. Preparation of GenoBaits DNA Probe Beads
a) Place GenoBaits Probe Beads at room temperature for 10 min before use;
b) Vortex for 15 seconds to mix well;
c) Prepare 50 μL of GenoBaits Probe Beads for each reaction, place them in a 0.2 mL EP tube;
d) Place the tube on a magnetic rack, allowing the beads to fully separate from the solution.
e) Remove the supernatant, retain the beads
f) Elution: For each reaction, add 150 μL of GenoBaits 1× Bead Wash Buffer, vortex for 10 seconds, transfer the tube to the magnetic rack, let the beads fully separate from the solution, and remove the supernatant.
g) Repeat step 6 above twice for a total of three washes.
6. Binding of hybridized fragments with GenoBaits DNA Probe Beads
a) Transfer the entire 16 μL of hybridization solution to the prepared beads
b) Vortex for 10 seconds to mix well, centrifuge for 5 seconds.
c) Place the EP tube in the PCR machine at 65° C. for 45 minutes, with a heat cover temperature of 75° C. (to bind DNA with the beads)
d) Shake for 5 s every 12 min.
7. Elution to remove unbound DNA (using 1× Wash Buffer from step 4)
a) Prepare a 65° C. elution buffer (completed on the PCR machine)
b) Prepare a room temperature elution buffer
c) Resuspend the beads, the suspension is used for step 8, and keep the remaining 10 μL as a backup.
8. PCR enrichment
a) According to the library type, prepare PCR reagents in a 0.2 mL PCR tube
b) Briefly vortex, centrifuge, and ensure the beads are still in the solution
c) Place the PCR tube in the PCR machine, with the heat cover temperature at 105° C., for PCR amplification
9. PCR Product Purification
a) Add 45 μL (1.5× volume) GenoPrep DNA Clean Beads to each PCR reaction, mix by vortexing, avoiding bubbles as much as possible; let stand for 5 min, and then briefly centrifuge to collect the liquid at the bottom of the tube.
b) Place the tube on a magnetic rack for at least 3 min until the solution is clear; remove the supernatant.
c) Keep the PCR tube on the magnetic rack, add 100 μL of 80% ethanol. Incubate at room temperature for 30 seconds, remove the supernatant.
d) Keep the PCR tube on the magnetic rack, and air-dry with the cap open for 10 minutes.
e) Remove the PCR tube from the magnetic rack, add 35 μL Tris-HCl, vortex to mix, let stand for 5 min, and then briefly centrifuge to collect the liquid at the bottom of the tube.
f) On the magnetic rack, wait until the solution clears (about 3 minutes), and transfer the supernatant to a new tube, store at −20° C.
g) The library requires further quality testing (e.g., concentration measurement and distribution assessment) for subsequent sequencing or the next step of the experiment.
10. Library testing
a) Measure the library with Qubit Fluorometer and Qubit dsDNA HS Assay Kit
b) Measure the average length of captured DNA library fragments on a digital electrophoresis system
c) Measure the library concentration with a KAPA Library Quantification Kit
11. Sequencing
Operate according to the requirements of the sequencing instrument. The average sequencing depth for target SNP loci is 105.88X.
Step 4: mSNP Genotyping
Genotypes for all mSNP loci are obtained according to the sequencing data processing workflow. The specific steps are as follows:
1. Use Trimmomatic software to remove adapters and low-quality reads.
2. Use BWA software to align the reads of each individual to the pig reference genome Sscrofa11.1 (GenBank: GCA_000003025.6; RefSeq: GCF_000003025.6, https://www.ncbi.nlm.nih.gov/datasets/genome/GCF_000003025.6). The pig reference genome Sscrofa11.1 was released by the Swine Genome Sequencing Consortium in 2017. The assembly was constructed using PacBio SMRT long-read sequencing data (approximately 65× coverage) from a female Duroc pig “TJ Tabasco (Duroc 2-14)”, with a total length of approximately 2.5 Gb across 20 chromosomes. As the reference individual is female, Y-chromosome sequences were incorporated from external resources and integrated into the assembly. Compared to the previous Sscrofa10.2 version, Sscrofa11.1 exhibits substantial improvements in assembly contiguity, local region sequencing and orientation, and gene model accuracy, and is widely used as the reference framework for pig genetics and genomics analysis.
3. Use SAMtools to generate BAM and sorted BAM files;
4. Use the GATK pipeline to generate a VCF file containing all mSNPs (including target SNP loci).

Example 3 demonstrates the operational process for using the liquid-phase chip of The present disclosure, while Example 4 evaluates the genotyping quality of the liquid-phase chip in samples from multiple pig farms.

### Example 4: Evaluation of Genotyping Quality of 50K mSNP Liquid-Phase Chip

Blood samples were collected from Duroc, Landrace, and Large White pigs from multiple farms. Genotyping was performed using the present disclosure as described in Example 3, to evaluate the stability of the detection and the quality of mSNP markers.

### 1. Stability

The stability of chip detection was generally measured by the consistency and correlation coefficient of the genotyping results from two tests of the same repeated sample. The genotyping consistency (0.992 (0.001)) and correlation coefficient (0.996 (0.001)) of the 60 repeated samples from the 50K mSNP liquid-phase chip for pigs were both greater than 99%, indicating good genotyping stability.

2. mSNP Quantity and Quality in Different Pig Populations

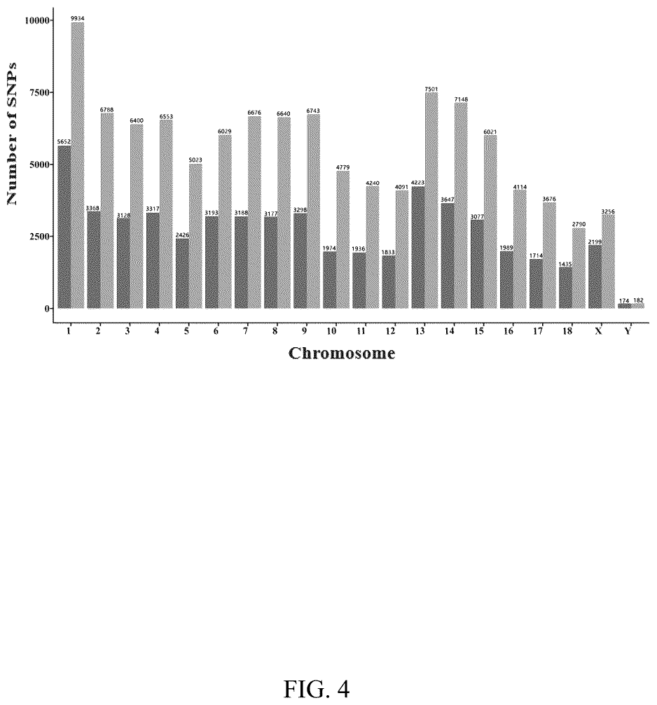

The 50K mSNP liquid-phase chip for pigs developed by the present disclosure has 52,000 target loci. After preliminary filtering of the sequencing data, the target loci were all detected as shown in Table 3. However, due to population differences (some populations had non-polymorphic mSNP loci), the number of detected mSNPs varied somewhat, as shown in Table 3. The three pig farms detected 52,000 target loci, with a total of 108,559-108,585 mSNPs detected, with slight differences but no significant variation. This indicates that the SNP loci designed by the present disclosure are universally applicable across different farms and can be widely used in practical populations. Additionally, as shown in FIG. 4, the number of mSNPs on each chromosome increased significantly.

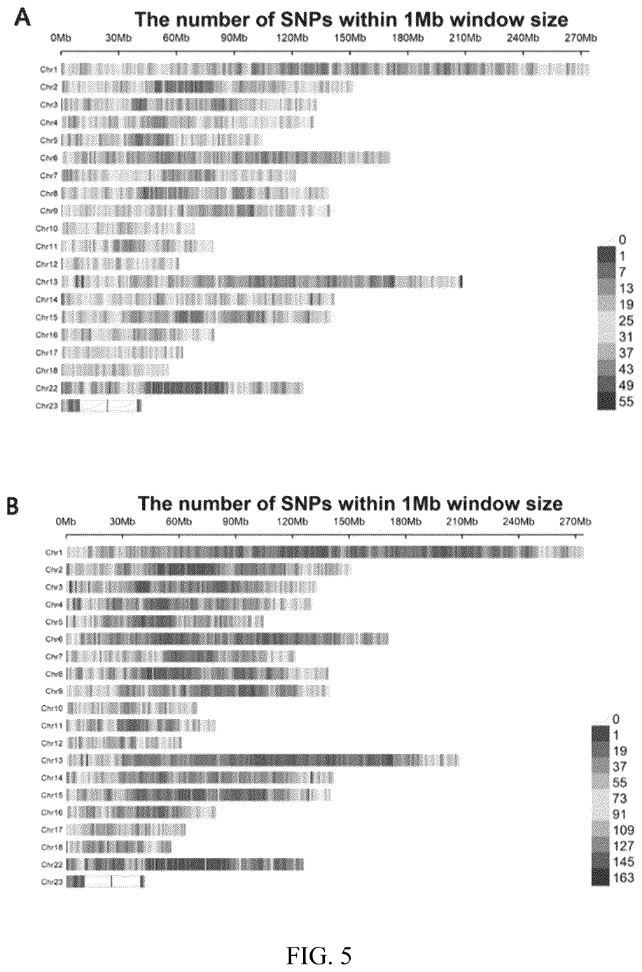

The density distribution of chromosomes is similar between the two, and the increase in mSNPs enhanced the SNP density without changing the general distribution of SNPs (FIG. 5), as shown in other pig farms as well. This indicates that in practical applications, the detection of mSNPs is consistent with the characteristics of multiple single nucleotide polymorphism detection technology, demonstrating that the mSNP detection technology of the present invention can effectively amplify the number of SNP markers, thereby improving the efficiency of fragment capture.

Moreover, the mSNP liquid-phase chip shows almost no difference between mSNPs (including target loci) and target loci in terms of missing rate and MAF, indicating that the amplification of SNP numbers by the mSNP liquid-phase chip does not reduce the quality of the chip data.

Table 3 shows the number of target loci and mSNPs before and after quality control for the 50K mSNP liquid-phase chip in three pig farms.

| | | | | Number of | Number of |
|---|---|---|---|---|---|
| | | Original | | Target Loci | Markers |
| | Number | Number of | Original | After | After |
| Pig | of | Target | Number of | Quality | Quality |
| Farm | Samples | Loci | Markers | Control | Control |
| Farm 1 | 534 | 52000 | 108585 | 43943 | 81461 |
| Farm 2 | 42 | 52000 | 108564 | 43998 | 89851 |
| Farm 3 | 97 | 52000 | 108559 | 43136 | 89167 |

### 3. Post-Quality Control Status of Genotypes in Different Populations

Genotype quality control (referred to as ‘QC’) is a routine operation after chip detection to ensure the quality of downstream analysis and is largely influenced by the population. In this example, the following QC steps were applied to multiple pig populations using the present disclosure:

Remove loci with unknown positions; remove SNPs with a call rate lower than 90%; remove SNPs with a minor allele frequency (MAF) lower than 0.05; remove SNPs with a significant deviation from Hardy-Weinberg equilibrium (P<10⁻⁶).

As shown in Table 3, a small number of SNPs were deleted after quality control for the 50K mSNP liquid-phase chip in the three pig farms. After quality control, there were 81,461 to 89,851 mSNP markers remaining, including 43,136 to 43,998 target loci. If the target loci did not meet the quality control standards, the mSNPs within the probe would also not meet the quality control criteria. After quality control, the number of mSNP markers did not decrease significantly, remaining approximately twice the number of target loci; this indicates that the present disclosure has selected high-quality SNPs upstream and downstream of the target loci, thereby increasing genomic information.

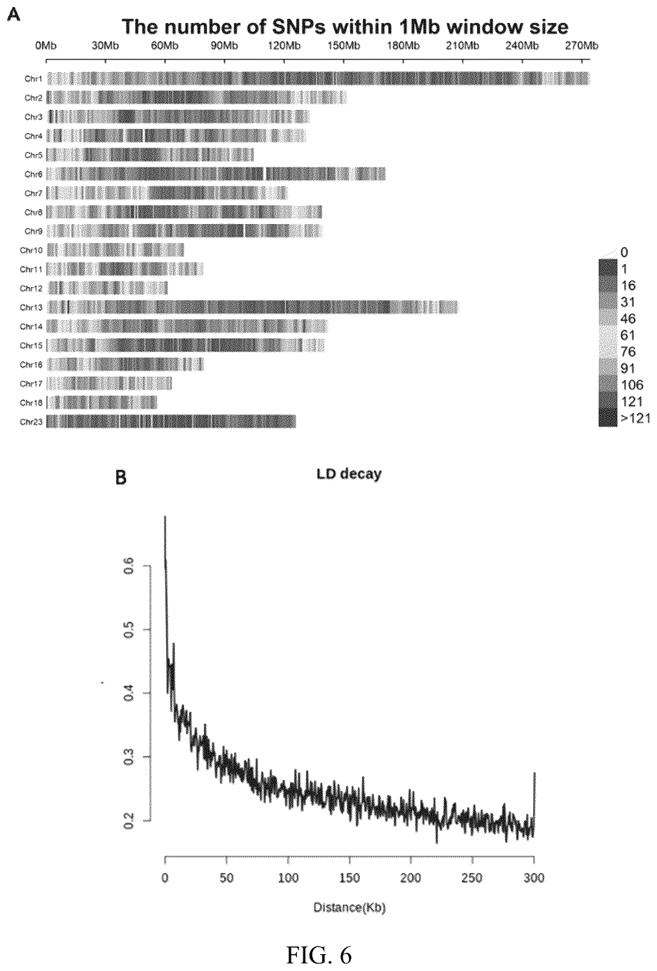

Taking Farm 1 as an example, as shown in FIG. 6, the data distribution after quality control of the mSNP liquid-phase chip did not change significantly, with the LD decay trend normal, but the average linkage disequilibrium (r2=0.45) was higher compared to using only target loci (r2=0.2), helping to improve the efficiency of genetic analysis and genomic selection. This indicates that after quality control, the mSNP can significantly increase the number of SNP detections without reducing data quality, which helps to retain more effective variations, thereby improving the efficiency of variation detection.

Example 4 evaluates the high stability and good genotype quality of the present disclosure, making the liquid-phase chip suitable for whole-genome association analysis and genomic selection. Example 5 takes genomic selection as an example to demonstrate the application effects of the present disclosure.

### Example 5: Application of the 50K mSNP Liquid-Phase Chip in Genomic Selection

After sampling 800 Large White pigs with growth and reproduction data, genotyping was performed using the present disclosure for genomic selection. These individuals also have genotype data from the solid-phase chip SNP chip Porcine GGP 50K (referred to as GGP50K, Neogen Corporation, USA).

### 1. Genotype Detection and Quality Control

Genotype quality control is an essential means to ensure the rationality of subsequent genetic analysis and molecular breeding results after genotyping is completed for all chips (including the present disclosure). In this example, the following quality control steps were applied sequentially:

1) Filter out multi-allelic SNPs; 2) Remove loci on sex chromosomes and loci with unknown positions; 3) Remove SNPs with a call rate lower than 90%; 4) Remove SNPs with a minor allele frequency (MAF) lower than 0.05; 5) Remove individuals with a call rate lower than 90%.

After quality control, all individuals were retained, and 88,105 mSNP loci of the present disclosure were retained, including 42,302 target SNP loci. The GGP50K chip retained 41,296 SNPs. The number of SNP markers on the GGP50K chip after quality control is close to the number of target SNP loci of the present disclosure.

### 2. Comparison of Genomic Selection Accuracy Between the Present Disclosure and GGP50K

Table 4 shows the comparison of genomic selection effects between the present disclosure and the mainstream chip GGP50K, with a similar number of markers on both chips. After quality control of genotypes, the 50K liquid-phase chip had 88,105 mSNP markers, including 42,302 target SNP loci, close to the number of 41,296 SNPs after GGP50K quality control. However, the genomic selection accuracy of GGP50K for three traits was lower than that of the present disclosure, with genomic selection accuracy lower by 1%-4% when using all mSNP markers (88,105) and lower by 1.8%-5.4% when using only target loci (42,302). The results indicate that the selection of target loci in the present disclosure ensures better genomic selection effects than GGP50K based on solid-phase chip technology. On the other hand, unlike solid-phase chips, the mSNP liquid-phase chip increased markers through multiple single nucleotide polymorphism technology. However, traditional single-marker analysis methods did not show the advantage of marker increase, with genomic selection accuracy slightly lower than using only 42,302 target loci.

It should be noted that genomic selection is currently the main method of molecular breeding, and its application effect is mainly measured by genomic selection accuracy. The accuracy of genomic selection for most traits is mainly improved by expanding the reference population (with both phenotypic and genotypic data) and evaluation methods. Expanding the reference population by one time means doubling the breeding cost, with accuracy only improving by 5-9%, and the improvement for some traits is even more challenging. The invention, under the same population size, has achieved an improvement in accuracy of the liquid-phase chip over GGP50K in some traits, equivalent to the effect of doubling the population size.

TABLE 4
Comparison of Genomic Selection Accuracy between
The present disclosure and Solid-Phase Chips

| | | Days to | 100 kg Live | Total |
|---|---|---|---|---|
| | Number | Reach 100 kg | Backfat | Number of |
| | of | Body Weight | Thickness | Piglets Born |
| SNP Type | Markers | (AGE) | (BF) | (TNB) |
| GGP50K | 41296 | 0.514 | 0.589 | 0.542 |
| Target loci | 42302 | 0.562 | 0.607 | 0.596 |
| mSNPs | 88105 | 0.554 | 0.599 | 0.588 |

Although Example 5 demonstrates that the present disclosure can be used for genomic selection and has advantages over solid-phase chips, the traditional single-marker analysis methods did not show the advantage of increasing the number of mSNP markers. The present disclosure proposes an improved mSNP analysis method, and Example 6 demonstrates its application in genomic selection. Likewise, the new method can also be used in whole-genome association analysis and other genetic analyses.

### Example 6: Application of the New mSNP Analysis Method in Genomic Selection

Example 5 demonstrates that although the mSNP liquid-phase chip increases the number of mSNP markers and has higher genomic selection accuracy than the GGP50K designed by Neogen in the United States, it does not show a marker quantity advantage compared to genomic selection using only target SNP loci. This is mainly due to the limitations of traditional analysis methods. Therefore, the present disclosure provides an mSNP utilization strategy based on haplotype analysis. This example illustrates the advantages of the new analysis method of the present disclosure.
1. The pig population, phenotypic data, and genotypic data are the same as in Example 5.
2. The genotype quality control standards and the number of individuals and markers after quality control are the same as in Example 5.
3. Haplotype block partitioning: the present disclosure provides a haplotype block partitioning strategy centered on target SNP loci. With the target SNP loci of the 50K liquid-phase chip prepared by the present disclosure as the center, the mSNPs within 200 bp upstream and downstream of the target loci are used as a haplotype block to construct haplotypes (named targeting block), and the number of targeting blocks is the same as the number of target loci after quality control.

### 4. Haplotype Allele and Genotype Matrix Construction

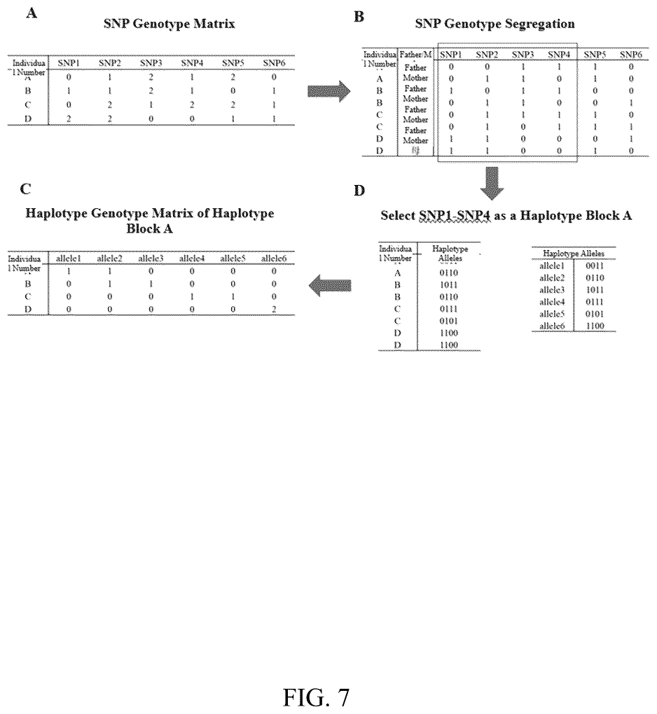

Within each targeting block, a haplotype matrix is constructed for all tested samples. As shown in FIG. 7, within each haplotype block (i.e., targeting block), individual haplotypes are re-encoded. In FIG. 7A, a genotype matrix for 4 individuals with 6 SNP markers is shown, where 0, 1, and 2 represent homozygous, heterozygous, and the other homozygous genotype, respectively. The four SNPs indicated by the red box in FIG. 7B form a targeted block. First, haplotype inference is performed based on SNP genotypes to obtain the paternal and maternal haplotypes for each individual within each haplotype block. After classifying all haplotypes, they are encoded as alleles. Then, for each haplotype allele within the haplotype block, individual diplotypes are encoded as 0, 1, or 2, representing the number of copies of a particular haplotype allele carried by the individual. As shown in FIG. 7C, after haplotype inference of the four SNPs, there are six haplotypes serving as alleles. Finally, an N-H matrix is generated, where N is the number of individuals, and H is the total number of haplotype alleles, as shown in FIG. 7D. After re-encoding the haplotypes of the 4 individuals according to the haplotype alleles, a 4*6 haplotype matrix is generated, where 4 represents the number of individuals, and 6 represents the number of haplotype alleles.

5. Genomic Selection Accuracy of the New mSNP Analysis Method of The present disclosure

Table 5 shows the effect of genomic selection for pig growth and reproductive traits using the new mSNP analysis method of the present disclosure. The present disclosure also compared the effectiveness of the targeted block with three other haplotype block partitioning methods. The 2 SNPs/block and 5 SNPs/block methods set 2 and 5 SNPs, respectively, as the size of the haplotype blocks, without overlap, for haplotype construction. 400 bp/block is the partitioning of blocks with a fixed 400 bp physical distance, non-overlapping, for haplotype construction. Among the four haplotype block partitioning methods, the targeted block proposed in the present invention achieves the highest genomic selection accuracy. Although the 400 bp/block method yields results similar to those of the targeted block, it requires traversing the entire genome, resulting in excessive computation time. In contrast, the targeted block selectively focuses on mSNPs near the target loci, significantly reducing computation time.

TABLE 5
Advantages of the New mSNP Analysis Method Proposed by
The present disclosure for Genomic Selection

| | Number of | | | |
|---|---|---|---|---|
| | Haplotype | Days to | 100 kg Live | Total |
| | Alleles | Reach 100 kg | Backfat | Number of |
| SNP/ | or SNP | Body Weight | Thickness | Piglets Born |
| Haplotype | Markers | (AGE) | (BF) | (TNB) |
| Target loci | 42302 | 0.562 | 0.607 | 0.596 |
| 2 SNPs/block | 153367 | 0.573 | 0.608 | 0.622 |
| 5 SNPs/block | 210348 | 0.567 | 0.604 | 0.616 |
| 400 bp/block | 238449 | 0.599 | 0.629 | 0.642 |
| Targeting | 240015 | 0.599 | 0.629 | 0.643 |
| block | | | | |

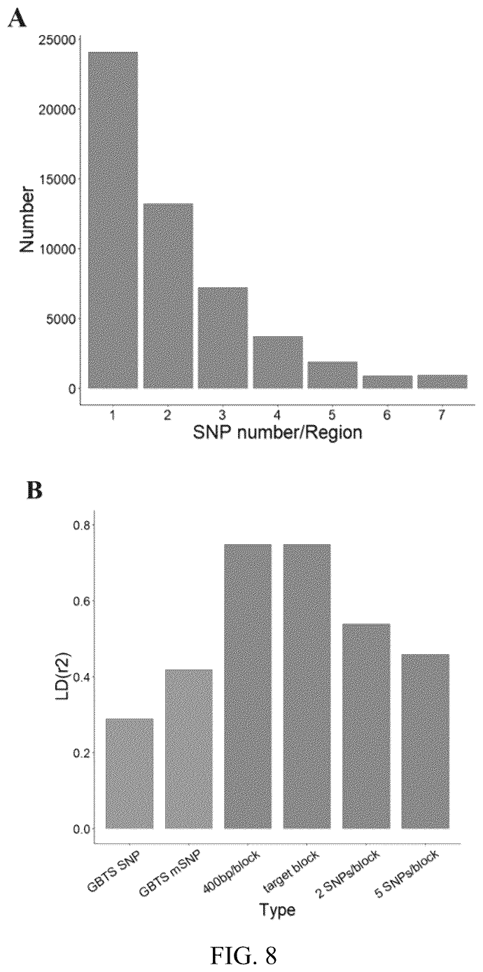

After quality control, the mSNP chip had 88,105 mSNP markers, including 42,302, forming 42,302 targeting haplotype blocks. As shown in FIG. 8A, 50% of the blocks contained more than 2 mSNPs (including target loci), adding 45,803 markers, increasing the number of markers. As shown in FIG. 8B, these mSNPs are in high linkage disequilibrium with the target loci (r²=0.75), but not in complete linkage disequilibrium. Therefore, they exhibit haplotype polymorphism and can provide more information than a single SNP (the average linkage disequilibrium between adjacent target loci is 0.3), resulting in higher genomic selection accuracy compared to using only the target loci. These applications are consistent with the design of the present disclosure, selecting mSNPs with medium to high linkage disequilibrium. Therefore, the mSNP liquid-phase chip can improve genomic selection accuracy through the haplotype analysis strategy proposed by the present disclosure without increasing application costs, that is, the new mSNP analysis method proposed by the present disclosure has the best genomic selection effect. This can also be extended to whole-genome association analysis and other genetic analyses.

What is claimed is:

1. A probe hybridization solution for genotyping pigs, comprising a set of isolated, synthetic DNA molecules, wherein the set of DNA molecules is configured to target a plurality of target single nucleotide polymorphism (SNP) loci, which are related to pig breeds.

2. The probe hybridization solution of claim 1, wherein the plurality of target SNP loci are identified by: aligning whole-genome sequencing data of Duroc, Large White, and Landrace pig breeds, selecting genomic regions with moderate linkage disequilibrium between markers, and screening and determining selected sites as the target SNP loci.

3. The probe hybridization solution of claim 1, wherein the genotyping quality of the target SNP loci meets the following criteria: a missing rate of NA<0.1, a minimum allele frequency (MAF)≥0.05, and heterozygosity (Het)<0.5.

4. The probe hybridization solution of claim 1, wherein the principles for selecting the target SNP loci are: (a) uniform distribution across chromosomes, with denser distribution at both ends of the chromosomes; (b) polymorphism considers MAF>0.35 in Duroc, Landrace, and Large White pigs; (c) average linkage disequilibrium (r²) with upstream and downstream SNP markers less than 0.85; (d) comparison with the QTLdb database, aiming for SNP markers to be located in QTL regions related to economic traits; (e) overlap with some loci of the known 50K chip for pigs.

5. The probe hybridization solution of claim 1, wherein the set of DNA molecules is determined with multiple single nucleotide polymorphism (mSNP) technologies, and each target SNP locus is targeted by 1-4 DNA molecules in the set of DNA molecules.

6. The probe hybridization solution of claim 1, wherein a length of each of the DNA molecules is 110 base pairs.

7. The probe hybridization solution of claim 1, wherein the set of DNA molecules comprises sequences set forth in SEQ ID NOs: 30,687, 30,697, 30,702, 30,711, 30,722, 30,725, 30,764, 30,773, 30,775, 30,782, 30,783, 30,799, 30,823, 30,855, 30,862, 30,887, 30,910, 30,911, 30,932, 30,933, 30,938, 30,959, and 30,960.

8. The probe hybridization solution of claim 1, wherein the set of DNA molecules comprises sequences set forth in SEQ ID NOs: 1-80,631.

9. The probe hybridization solution of claim 1, wherein a concentration of the DNA molecules in the probe hybridization solution is 1-5 pmol/ml.

10. The probe hybridization solution of claim 1, wherein the probe hybridization solution comprises a buffer solution, which is a mixture of EDTA and Tris-HCl.

11. The probe hybridization solution of claim 1, wherein for a total volume of 500 ml, the probe hybridization solution further includes:

| Component name | Quantity |
|---|---|
| Pooled, barcoded library | 0.6 μL |
| GenoBaits Block I | 5 μL |
| GenoBaits Block II for | 2 μL |
| ILM/MGI | |
| The DNA molecules | 300 ng |

12. The probe hybridization solution of claim 9, wherein the probe hybridization solution is concentrated to dryness using a vacuum concentrator at a temperature≤60° C.

13. A liquid-phase chip, comprising the probe hybridization solution of claim 1.

14. A screening method for pig breeding using the liquid-phase chip of claim 13, comprising:
(a) obtaining samples from the pigs to be tested and extracting genomic DNA;
(b) constructing pig cDNA libraries;
(c) hybridizing and sequencing the constructed libraries with the liquid-phase chip;
(d) performing mSNP genotyping according to the sequencing data operation process, and determining the genotypes of all liquid-phase chip marker loci for each individual.

15. The method of claim 14, wherein the mSNP genotyping comprises:
Step 1: after determining the genotypes of all mSNP markers for the individual liquid-phase chip, perform quality control on the mSNP genotypes; the quality control is carried out in the following order:
a) filter out multi-allelic variants;
b) remove sex chromosomes and loci with unknown positions;
c) remove SNPs with a call rate below 90%;
d) remove SNPs with a minor allele frequency (MAF) below 0.05; and
e) remove individuals with a call rate below 90%;
Step 2: using the target SNP loci as the core, define a 200 bp upstream and downstream region as a haplotype block, dividing the genome into 52,000 haplotype blocks, each with at least one mSNP marker, with varying numbers;
Step 3: for each haplotype block, infer haplotypes, determine haplotype alleles, and construct haplotype genotypes or diplotypes for each haplotype block in the tested sample, thereby constructing diplotype vectors for all haplotype blocks in the tested sample, similar to genotype vectors for all mSNP markers; and
Step 4: based on the diplotype vectors of all samples, apply genetic analysis or molecular breeding methods, with each haplotype block treated as a marker, and haplotypes within the block as alleles and diplotypes as genotypes.
